(19) United States
(12) Patent Application Publication
(10) Pub. No.: US 2026/0367054 A1
(43) Pub. Date: Nov. 19, 2026

(54) ASSEMBLY AND METHOD FOR MONITORING A SYSTEM FOR AMMONIA EMISSIONS

(71) Applicant: Drager AG & Co. KGaA, Lubeck (DE)

(72) Inventors: Andreas Nauber, Lûbeck (DE); Norbert Wruck, Lúbeck (DE); Felix Watge, Lübeck (DE)

(21) Appl. No.: 19/369,448

(22) Filed: Oct. 27, 2025

(30) Foreign Application Priority Data
Oct. 28, 2024 (DE) 102024131389.7
May 23, 2025 (DE) 102025120244.3

(51) Int. Cl. G01N 33/00

(52) U.S. Cl. CPC G01N 33/0054

(57) ABSTRACT

The present disclosure relates to a monitoring assembly and a monitoring method that are configured to monitor a system for ammonia emissions. The monitoring assembly comprises at least one monitoring unit and a central computer. A predetermined upper limit value for the ammonia emissions of the system is stored in a data memory of the monitoring unit. An ammonia sensor of the monitoring unit measures the ammonia content in an emitted gas mixture. A communication unit of the monitoring unit generates a message comprising the stored upper limit value and the or each measured ammonia content. This message is transmitted to the central computer. An evaluation unit of the central computer decides whether the actual ammonia emissions of the system are below the transmitted upper limit value or not.

### CROSS-REFERENCE TO RELATED APPLICATIONS

This application claims the priority of German Patent Application No. 102024131389.7, filed on Oct. 28, 2024, and titled “ASSEMBLY AND METHOD FOR MONITORING A SYSTEM FOR AMMONIA EMISSIONS”, and the priority of German Patent Application No. 102025120244.3, filed on May 23, 2025, and titled “ASSEMBLY AND METHOD FOR MONITORING A SYSTEM FOR AMMONIA EMISSIONS”; each of which is hereby incorporated by reference in its entirety for all nonlimiting purposes.

### TECHNICAL FIELD

The present disclosure relates to a monitoring assembly and a monitoring method that are configured to monitor at least one system for ammonia emissions.

### BACKGROUND

One possible application of such a monitoring assembly and such a monitoring method as described herein is as follows: At an agricultural enterprise, livestock are bred, whereby ammonia (NH₃) is generally formed and emitted. The ammonia emissions from this agricultural enterprise should not exceed a predetermined upper limit value. Typically, the upper limit value relates to a cumulative emission in a given reference time period, but can also specify an instantaneously emitted amount of ammonia.

The present disclosure is based on the object of providing a monitoring assembly and a monitoring method that, in a more flexible manner than known monitoring assemblies and monitoring methods, allow at least one system to be remotely monitored for compliance with at least one predetermined upper limit value for ammonia emissions.

### SUMMARY

The object of the present disclosure is achieved by a monitoring assembly having the features described herein. Advantageous embodiments are specified in the claims. Advantageous embodiments of the monitoring assembly according to the present disclosure are, as far as appropriate, also advantageous embodiments of the monitoring method according to the present disclosure, and vice versa.

The monitoring assembly according to the present disclosure and the monitoring method according to the present disclosure are configured to monitor a first system for ammonia emissions, optionally additionally at least one second system, the or every second system being different from the first system, in particular three or more systems. The or each monitored system emits ammonia permanently or at least temporarily or can emit ammonia at least temporarily.

At least one first upper limit value is predetermined for the first monitored system. It is required that the actual ammonia emissions of the first system do not exceed the first upper limit value. If a second system is monitored, at least one second upper limit value is predetermined for this second system. The first upper limit value can be different from the second upper limit value. Optionally, at least two upper limit values are predetermined for a monitored system. The or each upper limit value predetermined for a system specifies an upper limit for the permissible ammonia emissions from that system.

In some examples, the or at least one upper limit value specifies an upper limit for the maximum amount or volume of ammonia that this system is permitted to emit during a predetermined reference time period, i.e., for a cumulative amount or a cumulative volume flow over the reference time period. In some examples, the or an upper limit value specifies an upper limit for the instantaneous or reference time period-averaged volume flow or mass flow of ammonia or an upper limit for the instantaneous or reference time period-averaged concentration of ammonia in an emitted gas mixture, in particular the concentration in ppm (parts per million).

Different upper limit values for the same system can relate to different reference time periods, for example a larger upper limit value can relate to a shorter reference time period and a smaller upper limit value can relate to a longer reference time period. It is also possible that a first upper limit value relates to an instantaneous emission and a second upper limit value for the same system relates to a cumulative emission during a reference time period. It is also possible that different upper limit values are predetermined for the same system for different seasons or times of day or modes of use of the system.

A first monitoring unit is assigned to the first monitored system. If at least one second system is monitored, a second monitoring unit is assigned to the or each second system. The or each monitoring unit belongs to the monitoring assembly according to the present disclosure. In the following, reference is made to “the monitoring unit,” and this refers to both the first monitoring unit and the or each optional additional monitoring unit, unless expressly specified otherwise.

The or each monitoring unit comprises at least one ammonia sensor. In one embodiment, at least one monitoring unit comprises at least two ammonia sensors. The term “first ammonia sensor” refers to an ammonia sensor of the first monitoring unit, and the term “second ammonia sensor” refers to an ammonia sensor of the second monitoring unit. This term can also refer to any one of a plurality of ammonia sensors in the same monitoring unit.

The or each ammonia sensor is configured to measure, at a respective particular measuring position, the ammonia content in a gas mixture that exits the monitored system, for example in [ppm] (parts per million) or vol. % or wt. % or in mass flow or volume flow. It is possible that at least one system comprises a plurality of ammonia sensors that measure the particular ammonia content at different measuring positions. Different ammonia sensors can use the same measuring principle or at least two different measuring principles. In some examples, the or each ammonia sensor repeatedly measures the ammonia content and is configured to provide a temporal course of the ammonia content.

Note: The wording that a sensor is configured to measure a physical variable means the following: The sensor directly measures the physical variable or another variable that correlates with the physical variable to be measured and is therefore a measure of the physical variable to be measured. The physical variable to be measured is, for example, the concentration of a target gas in a gas sample, in this case that of ammonia; the sensor operates as a photoelectric sensor; the target gas attenuates electromagnetic radiation in a wavelength range dependent on the target gas; a detector measures the intensity of incident electromagnetic radiation; and the other, correlating variable is the measured intensity of the incident electromagnetic radiation. The measurement provides at least one value for the physical variable sought.

Furthermore, the or each monitoring unit comprises a data memory. The first monitoring unit therefore comprises a first data memory; the optional second monitoring unit comprises a second data memory. The first data memory contains at least the following information about the first system in a computer-readable form:
the or at least one, preferably each first upper limit value, the or each stored first upper limit value being predetermined for the first system, i.e., for the system monitored by the first monitoring unit, and
a unique identifier of the ammonia sensor of the first monitoring unit, in the case of several ammonia sensors a respective unique identifier.

Accordingly, the optional second data memory stores the or each second upper limit value and a respective unique identifier of the or each ammonia sensor of the second monitoring unit. The or each second upper limit value is predetermined for the second system.

The unique identifier distinguishes an ammonia sensor from the or any other ammonia sensor in the monitoring assembly. In one embodiment, a unique identifier of the monitoring unit and/or the monitored system and a locally unique identifier of the ammonia sensor are stored in the data memory of a monitoring unit. The unique identifier of the monitoring unit distinguishes this monitoring unit from the or any other monitoring unit in the monitoring assembly. The unique identifier of the system distinguishes this system from any other system monitored by this monitoring assembly. The locally unique identifier of the ammonia sensor distinguishes this ammonia sensor at least from any other ammonia sensor of the same monitoring unit, but not necessarily from any ammonia sensor or other sensor of a different monitoring unit.

Preferably, the data memory is protected against unauthorized external access, but at a minimum against unauthorized write access. This avoids, or at least greatly reduces the risk of, a stored upper limit value being changed, in particular increased, without authorization. Preferably, however, an authorized person can overwrite and thereby change a stored upper limit value and optionally other stored information using a suitable device. Preferably, this authorized person has previously successfully authenticated themselves (successful authorization check). Many suitable methods and devices for protection against unauthorized modification of stored data and for authorization checks are known from the prior art.

According to the present disclosure, the first monitoring unit further comprises a first signal-processing communication unit. The optional second monitoring unit comprises a second signal-processing communication unit. The communication unit of a monitoring unit is at least temporarily in data communication with the or each ammonia sensor of this monitoring unit, preferably in wireless data communication via radio waves, in some examples instead or additionally in wired data communication.

The first communication unit has at least temporary read access to the first data memory, i.e., the data memory of the first monitoring unit. The first communication unit is configured to generate a message at least once, preferably a plurality of times. The message or at least one message, optionally each message, comprises the following information:
for the or each ammonia sensor of the first monitoring unit, at least one measured ammonia content, in particular a measured ammonia concentration, that this ammonia sensor has measured, preferably including the measurement unit, and the stored unique identifier of this ammonia sensor and
the or at least one, in one embodiment each, first upper limit value stored in the data memory.

It is possible that a message from the first communication unit comprises a plurality of ammonia contents that were measured at different times, preferably together with a time stamp for each measured value. If a plurality of upper limit values is predetermined for the first system, the message preferably comprises the or each limit value applicable at the time of measurement, for example a limit value applicable for that time of year or time of day.

It is possible for a first message to comprise at least one measured ammonia content and at least one stored first upper limit value and for at least one further message, which has also been generated by the first communication unit, to likewise comprise at least one measured ammonia content but no upper limit value.

The foregoing applies mutatis mutandis to the second communication unit.

The monitoring assembly according to the present disclosure further comprises a central computer. The central computer is located at a distance from the or each monitoring unit and at a distance from the or each monitored system. The central computer can be a stationary device and/or located “in the cloud” or a combination of a stationary device and a computer “in the cloud.” The central computer comprises a signal-processing evaluation unit. The evaluation unit can comprise software that can be executed on a processor of the central computer. The evaluation unit can also be implemented as a signal-processing unit. In some examples, the central computer is a stationary or mobile data processing device, for example a workstation computer or a portable computer, in particular a smartphone.

The monitoring assembly is configured to establish, at least temporarily, a data connection between the or each communication unit belonging to the or a monitoring unit of the monitoring assembly and the central computer. The monitoring assembly is configured to cause the or each message generated by the first communication unit to be transmitted to the central computer via a data connection between the first communication unit and the central computer. Optionally, a message can be transmitted in the reverse direction to the first communication unit via this data connection, for example a query message or a confirmation message. The same applies to the second communication unit and the central computer. In some examples, the or each message is transmitted to the central computer in such a way that it is protected against unauthorized modification, in particular by encrypted transmission and/or by a suitable signature.

The evaluation unit is configured to automatically decide the following for the first system: whether the actual ammonia emissions of the first system do not exceed the or an upper limit value for the first system, this first upper limit value specifying the maximum permissible ammonia emissions and having been transmitted as part of a message from the first system to the central computer. In other words: Does the first system comply with the or each currently valid first upper limit value or not?

For this decision, the evaluation unit uses the or at least one, preferably every message that has been transmitted from the first communication unit to the central computer. The message used comprises the or at least one ammonia content measured by the or an ammonia sensor of the first monitoring unit, as well as the or at least one first upper limit value stored in the first data memory.

As already explained above, the following example is possible: The transmitted first message comprises at least one measured ammonia content and at least one stored first upper limit value. At least one further message transmitted also comprises at least one measured ammonia content, but no upper limit value. The evaluation unit uses the upper limit value transmitted as part of the first message until another message from the first monitoring unit also comprises an upper limit value.

If a second system is monitored, the evaluation unit can decide accordingly for the second system.

The monitoring method according to the present disclosure is carried out using a monitoring assembly according to the present disclosure and comprises the corresponding steps.

According to the present disclosure, the first monitoring unit and optionally the or each further monitoring unit each comprise at least one ammonia sensor. Such an ammonia sensor is preferably located in or on the system to be monitored and therefore measures the ammonia content on site. The values measured on site are transmitted to the remote central computer. Therefore, the first system and optionally a further system can be monitored relatively reliably from a distance even if another system in the vicinity of a system monitored according to the present disclosure also emits or can emit ammonia or if wind blows away an emitted gas mixture.

Often, legal and/or regulatory requirements require that a system does not emit more ammonia than specified by an upper limit value for that system. Such upper limit values for ammonia emissions are often established, in particular because ammonia in large quantities can be harmful to living organisms or is, at a minimum, perceived as a nuisance. The present disclosure makes it easier to monitor compliance with such limit values.

The present disclosure makes it possible for the or each monitored system to be monitored remotely, namely by a user of the central computer. In some examples, the data connection between the first monitoring unit and the central computer is established at least in part using a public data connection network, in particular a wired network or a mobile network. By virtue of the present disclosure, it is, in particular, not necessary for a person to be permanently on site to monitor the ammonia emissions of a system. As a general rule, it is merely necessary for a person to monitor and, if necessary, repair the or each ammonia sensor in or on this system from time to time.

The present disclosure can be implemented in combination with a procedure in which a list is kept in computer-evaluable form on the central computer or even in paper form in a central office, said list containing the or each upper limit value of the or each monitored system. With this combination it is possible to compare the upper limit value that was transmitted to the central computer as part of the message with a stored upper limit value.

However, the present disclosure eliminates the need to use such a list. Furthermore, the present disclosure eliminates the need to keep such a list constantly up to date. Drawing up and maintaining such a list is often relatively complex, in particular because an upper limit value can differ from system to system, it can depend on the season, the time of day and/or the current mode of use, and it can change with changing legal and/or regulatory requirements. By virtue of the present disclosure, it is sufficient to store the or each upper limit value for a monitored system in the data memory of the monitoring unit and to maintain it continuously. According to the present disclosure, the or each stored and maintained upper limit value is transmitted to the central computer and received and processed by the evaluation unit.

The present disclosure can be used in combination with an example in which the ammonia sensor of the first monitoring unit compares a measured ammonia content with an upper limit value and the ammonia sensor itself, or the monitoring unit generates an alarm if the measured ammonia content exceeds the upper limit value. This alarm is output, for example, in or on the first system in at least one form that can be perceived by a human. However, the present disclosure eliminates the need for the first monitoring unit to perform this comparison itself and output the alarm. It is possible, but by virtue of the present disclosure not necessary, for an alarm unit to be present on site. Rather, it is sufficient that an ammonia sensor is configured to measure the ammonia content and that the measured ammonia content is transmitted to the central computer together with the stored upper limit value. It is possible, but by virtue of the present disclosure not necessary, for the first communication unit to comprise the message in such a way that the message additionally contains the information indicating that the measured ammonia content exceeds the upper limit value. The decision is made by the evaluation unit.

According to the present disclosure, the evaluation unit automatically decides whether the actual ammonia emissions of the first system do not exceed the stored and transmitted first upper limit value. In the case of a plurality of monitored systems, the evaluation unit preferably decides this for each monitored system. As already explained, the upper limit values can vary from system to system. Preferably, if the actual ammonia emissions of a monitored system exceed the or at least one upper limit value predetermined for this system, the evaluation unit has the following effect: A notification is output on an output unit in at least one form perceivable by a human. This notification contains the information indicating that the ammonia emissions are above the upper limit value and preferably an identifier of the system concerned, an identifier of the measured ammonia emissions and/or an identifier of the upper limit value. The output unit can be part of the central computer or can be spatially spaced apart from the central computer and the or each monitored system. It is possible that the alarm is also output on an output unit belonging to the system in question.

According to the present disclosure, the first communication unit generates a message at least once. The or each message generated by the first communication unit comprises information about an ammonia content measured by the first ammonia sensor and a unique identifier of the first ammonia sensor. It is possible that the first monitoring unit comprises a plurality of first ammonia sensors and the message or at least one message for each first ammonia sensor comprises information about an ammonia content measured by this ammonia sensor. In some examples, this message additionally comprises a unique identifier of the monitored system and/or a unique identifier of the monitoring unit. In these examples, it is sufficient for the identifier of an ammonia sensor to distinguish this ammonia sensor from any other ammonia sensor of the same monitoring unit, i.e., is a locally unique identifier. Preferably, the message additionally comprises a time stamp for each measured value, the time stamp specifying the time of the measurement. It is also possible for the generated and transmitted message to comprise an identifier of the monitored system, the identifier of the monitored system serving as an identifier of the ammonia sensor.

Preferably, the first communication unit repeatedly generates a message, for example with a fixed sampling rate. In some examples, each message additionally comprises the first upper limit value predetermined for the first monitored system and stored in the first data memory.

In some examples, a first message comprises at least one upper limit value for the first monitored system, preferably each first upper limit value, while at least one further message comprises at least one measured ammonia content, but not necessarily the or each first upper limit value. The evaluation unit uses the first upper limit value or each first upper limit value that was transmitted to the central computer as part of the first message for the first system until a different upper limit value is transmitted to the central computer as part of a further message for the first system. In other words, once an upper limit value has been transmitted for the first system, it will be used until an upper limit value is transmitted again for the first system. In addition, the evaluation unit preferably uses at least the most recent ammonia content measured by the or an ammonia sensor of the first monitoring unit, optionally a time stamp of the measurement. To decide whether or not the first upper limit value is complied with, the evaluation unit uses the or each ammonia content having been measured and transmitted as part of a message to the central computer.

It is also possible for the evaluation unit to cause, by sending a corresponding request message to the communication unit, the following step: The communication unit transmits a message with the or each stored first upper limit value to the evaluation unit.

The foregoing applies mutatis mutandis to a second monitoring unit having at least one second ammonia sensor, the or each second ammonia sensor measuring the ammonia content emitted by a second monitored system, and having a second communication unit that generates a message. The generated message comprises an ammonia content measured by the second ammonia sensor and a second upper limit value valid for the second system.

According to the present disclosure, the first ammonia sensor is configured to measure the content of ammonia in a gas mixture that has been or is emitted by the first system. In some examples, the first ammonia sensor measures the concentration (proportion) of ammonia in the gas mixture as ammonia content, for example measured as ppm. In some examples, the first monitoring unit comprises a volume flow sensor. The volume flow sensor is configured to measure the volume flow or mass flow of the gas mixture from the first system. The volume flow is the volume per unit time of said gas mixture; the mass flow is the mass per unit time. The first communication unit or the evaluation unit derives the volume flow or mass flow of ammonia from the first system from the measured ammonia concentration and the measured volume flow or mass flow of the gas mixture.

According to the present disclosure, a first upper limit value is transmitted to the central computer as part of a message for the first monitored system. In some examples, a reference time period and a monitoring period are predetermined. The monitoring period is longer than the reference time period, preferably at least twice as long, in particular at least ten times as long. The first upper limit value for the first system specifies an upper limit for the amount of ammonia, the first system being permitted to emit a maximum of this amount of ammonia in total or on average during the reference time period.

One or more examples are described below. The evaluation unit determines, for the first monitored system and for a plurality of periods, the amount of ammonia that the first system actually emits during this period. Each monitored period has a duration equal to or greater than the reference time period and lies within the monitoring period. The evaluation unit determines each error period in the monitoring period. An error period is at least as long as the specified reference time period. In an error period, the amount of ammonia emitted per reference time period is consistently greater than the first upper limit value. The duration of an error period is preferably not specified, but results from the monitoring and evaluation just described. Of course, it is possible that the evaluation unit detects that no error period occurred during the monitoring period, which is usually the desired result.

In some examples, the evaluation unit causes information about the determined error periods to be output in at least one form perceivable by a human. For example, a graphical representation with a time axis illustrates the error periods. For example, the graphical representation shows the temporal course of ammonia emissions, depending on the repeatedly measured transmitted ammonia content. Furthermore, the graphical representation shows the error periods and preferably the first upper limit value. This representation allows a user to quickly determine whether the first system is operating correctly or whether, and if so when, it is emitting too much ammonia.

The present disclosure makes it possible, in particular, to remotely monitor an exhaust gas purification system. Said exhaust gas purification system filters ammonia from a gas mixture emitted by the first monitored system before the gas mixture enters the environment. During an error period, the exhaust gas purification system may not be operating correctly or may be switched off.

The examples described below reduce the risk of manipulation as well as the risk of error. According to these examples, a computer-evaluable description of a geoposition region is specified, i.e., a specific region on the earth's surface. For example, the description specifies a certain geoposition and a radius and thus a circle around the geoposition. Or the description specifies the geoposition of the n vertices of an n-gon. The geoposition region is defined as follows: Each location of the first monitored system has a geoposition that lies within the geoposition region as described by the description.

According to the present disclosure, the first monitoring unit comprises a first geoposition sensor. The first geoposition sensor is configured to measure the current geoposition of the first ammonia sensor. For example, the first geoposition sensor measures its own geoposition and is positioned sufficiently close to the first ammonia sensor. If the first monitoring unit comprises a plurality of first ammonia sensors, in some examples the first geoposition sensor is configured to measure the relevant geoposition of each first ammonia sensor. Or the same geoposition is measured and used for each ammonia sensor of the first monitoring unit. In some examples, each first ammonia sensor is assigned its own geoposition sensor.

According to the present disclosure, the first communication unit generates a message. This message comprises a measured ammonia content and is transmitted to the central computer. According to the embodiment just described, the transmitted message additionally comprises the geoposition of the first ammonia sensor that measured this ammonia content. In the case of a plurality of first ammonia sensors, the message comprises, in one embodiment, the common geoposition or the relevant geoposition of each first ammonia sensor.

The evaluation unit is configured to automatically check the following: Is the measured geoposition that was transmitted to the central computer as part of the message within the geoposition region? For this check, the evaluation unit uses the stored description of the geoposition region.

It is possible that the first message contains the measured geoposition of the first ammonia sensor, but at least one further message does not contain this geoposition. The evaluation unit uses the geoposition transmitted as part of the first message until another message also comprises a measured geoposition of the first ammonia sensor.

In one or more examples, the evaluation unit has at least temporary read access to a central data memory that is spatially spaced apart from the or each monitoring unit and preferably spatially spaced apart from the or each monitored system and preferably belongs to the central computer. This data memory contains the computer-evaluable description of the geoposition region in which the first monitored system is located. If a plurality of systems is monitored, a description of a geoposition region in which the monitored system is located is preferably stored in the central data memory for each monitored system.

In some examples, the need to provide such a central data memory is avoided. Rather, a computer-evaluable description of a geoposition region in which the first system is located is stored in a data memory of the first monitoring unit, for example in the first data memory. This stored description is transmitted to the central computer together with the measured ammonia content and the measured geoposition of the ammonia sensor and used by the evaluation unit. If a plurality of messages is transmitted successively from the first communication unit to the central computer, at least one message, preferably the first message transmitted, comprises the description of the geoposition region. The transmitted description of the geoposition region is preferably used until a description of the geoposition region in which the first system is located is transmitted again.

If a second system is monitored, a computer-evaluable description of a second geoposition region is also stored in the central data memory, the second system being located in the second geoposition region.

A combination is also possible: A description of the first geoposition region is stored in the central data memory, and the transmitted message comprises a description of the first geoposition region. On the one hand, this combination creates redundancy. On the other hand, the evaluation unit can check whether the stored description matches the transmitted description of the first geoposition region or not. A stored description can be out of date.

It is possible that the evaluation unit detects the following result: The first ammonia sensor or a first ammonia sensor is not located in the first geoposition region, i.e., not in the region in which the first system is located. In many cases, this is an indication that this ammonia sensor is not located in or on the monitored first system, but further away. This fact, in turn, can lead to the actual ammonia emissions of the first system not being measured correctly, in particular to emissions being measured that are too low.

Preferably, in response to the detection of the event that a first ammonia sensor is located outside the geoposition region, the evaluation unit causes the following: A corresponding notification is generated and output in at least one form perceivable by a human. This notification preferably identifies the first system, the geoposition region in which the first system is located, and the measured geoposition of the first ammonia sensor outside this geoposition region. Preferably, this notification is output on an output unit of the central computer. The output of this notification allows a user to check whether the ammonia sensor is actually incorrectly positioned or whether it is capable of correctly measuring the ammonia content of the first system even outside the geoposition region.

According to the present disclosure, the first communication unit generates a message. This message comprises an ammonia content measured by the first ammonia sensor and the first upper limit value stored in the first data memory. In some examples, the first data memory is protected against unauthorized write access. The examples described below can be used in combination with such protection. In many cases, such a combination further increases protection against manipulation. In addition, these examples make it possible in many cases to detect a technical error, which leads to an incorrect upper limit value being transmitted.

According to one or more examples, the central computer has at least temporary write access and also read access to a central database. The central computer creates and manages a data record in this central database for the or each monitored system. The data record for the first monitored system comprises each first upper limit value transmitted as part of a message from the first communication unit to the central computer. Preferably, the data record additionally comprises a time stamp, the time stamp indicating the point in time at which the message with the first upper limit value reached the central computer. The same applies to the data record that is created and updated for a second monitored system.

The feature that the first upper limit value is transmitted as part of the message from the first communication unit to the central computer allows the following: A user or a data-processing evaluation unit performs a plausibility check. For example, a general upper limit value is predetermined that applies to each system to be monitored or at least to all monitored systems located in a specific area. This general limit value may vary depending on the season and/or the time of day. If the or a transmitted first upper limit value is above this general upper limit value, there is a possibility that a technical error or manipulation has occurred.

In some examples, the first communication unit successively transmits a plurality of messages to the central computer, at least some of these messages comprising the stored first upper limit value. Over time, this embodiment leads to a temporal sequence of transmitted first upper limit values, these first upper limit values being or having been stored in the first data memory and having been stored in the data record for the first system. This temporal sequence can also be checked for plausibility. A strong variation in the first upper limit values in the temporal sequence can be correct, for example if the first upper limit values vary over the season or time of day or if legal or regulatory requirements change. However, a strong variation can also be an indication of a technical error or manipulation.

One or more examples described below make it possible in particular to check each data connection of the monitoring assembly. According to these examples, the monitoring assembly additionally comprises a first signal transmitter. If the first monitoring unit comprises a plurality of ammonia sensors, the monitoring assembly preferably comprises a first signal transmitter for each first ammonia sensor. The or each first signal transmitter is configured to acquire a content value for an ammonia content, this content value having been specified to said signal transmitter by a user and/or by the evaluation unit or another component of the monitoring assembly. In an alternative, the or at least one signal transmitter has measured the content value in a gas sample. The or each first signal transmitter is configured to generate a signal that comprises information about the value for an ammonia content that has been specified to this signal transmitter and acquired by the signal transmitter. In one embodiment, the generated signal further comprises an identifier of the signal transmitter, which identifier distinguishes said signal transmitter from the or any other signal transmitter and from any ammonia sensor of the first monitoring unit.

According to one or more examples, the monitoring assembly can be operated either in a monitoring mode or in a checking mode. According to these examples, the monitoring method according to the present disclosure comprises the step of carrying out a checking method at least once. The monitoring assembly is checked when operating in the checking mode and through the checking method.

The or each first ammonia sensor is used to monitor the first system when operating in the monitoring mode. When operating in the checking mode, however, the first signal transmitter is used, in particular to check the data connections to the central computer. If the first monitoring unit comprises a plurality of ammonia sensors, a first signal transmitter is used for each first ammonia sensor. In one embodiment, the or each first ammonia sensor is replaced by the or a signal transmitter. It is also possible that either an ammonia sensor or a signal transmitter is activated, depending on the mode in which the monitoring assembly is currently operating.

When operating in the checking mode, the monitoring assembly is configured to perform the following steps, and the checking method comprises the following steps:
The or each first signal transmitter generates a signal. This signal comprises information about the value of the ammonia content that has been specified to this signal transmitter and acquired by the signal transmitter.
The particular signal from each first signal transmitter is transmitted to the central computer and from there to the evaluation unit.
The evaluation unit determines, for the or each signal transmitter of the first monitoring unit, which value of an ammonia content is specified to this signal transmitter. For this purpose, the evaluation unit uses the transmitted and received signal of the first signal transmitter, or in the case of a plurality of first ammonia sensors, the transmitted and received signals of the first signal transmitters.
Preferably, the evaluation unit is given the value that is specified to a signal transmitter. The evaluation unit compares the specified value with the value that the evaluation unit has determined using the transmitted signal.
Preferably, the signal from the signal transmitter additionally comprises a time stamp, the time stamp indicating when the signal was sent. Using this time stamp, the evaluation unit determines how long it took for the signal to be transmitted from the first signal transmitter to the central computer.

The present disclosure makes it particularly easy to detect the following possible errors and faults:
A data connection between an ammonia sensor and thus a signal transmitter on the one hand and the evaluation unit on the other hand is interrupted or otherwise disturbed.
Signal processing on the way from an ammonia sensor and therefore from a signal transmitter to the evaluation unit is operating incorrectly.
A component of the monitoring assembly is not supplied with sufficient electrical energy.

In one or more examples, at least one signal transmitter, preferably each signal transmitter, is provided with a temporal course of values of an ammonia content. In other words: At least one signal transmitter is given different values for an ammonia content, one value after the other. In one embodiment, each signal transmitter is given a test pattern with values for the ammonia content. In some examples, the temporal course comprises, on the one hand, the value zero and, on the other hand, the maximum possible value for an ammonia content that the respectively replaced ammonia sensor is still capable of measuring and/or that can occur in reality. The maximum value is preferably between 90% and 100%.

On the one hand, one or more embodiments with the predefined temporal course make it easier to distinguish between the monitoring mode and the checking mode, in particular if the predefined temporal course cannot occur in reality during operation of the monitored system. On the other hand, these embodiments make it easier to detect the following faults, additionally or better than without this embodiment:
For some ammonia content values, signal processing is operating incorrectly.
The step of transmitting a signal from the signal transmitter and thus from the replaced ammonia sensor to the evaluation unit is subject to a considerable delay.

The monitoring assembly according to the present disclosure and the monitoring method according to the present disclosure are configured to monitor a first system and optionally at least one further system. Possible applications of the present disclosure are listed below by way of example.

The or a monitored system is, for example, an agricultural enterprise, in particular a holding where pigs and/or other domestic animals are bred. The or at least one system may also be a waste incineration system or a recycling system or a wastewater treatment system.

In the following, the present disclosure is described on the basis of an exemplary embodiment. In the drawings,

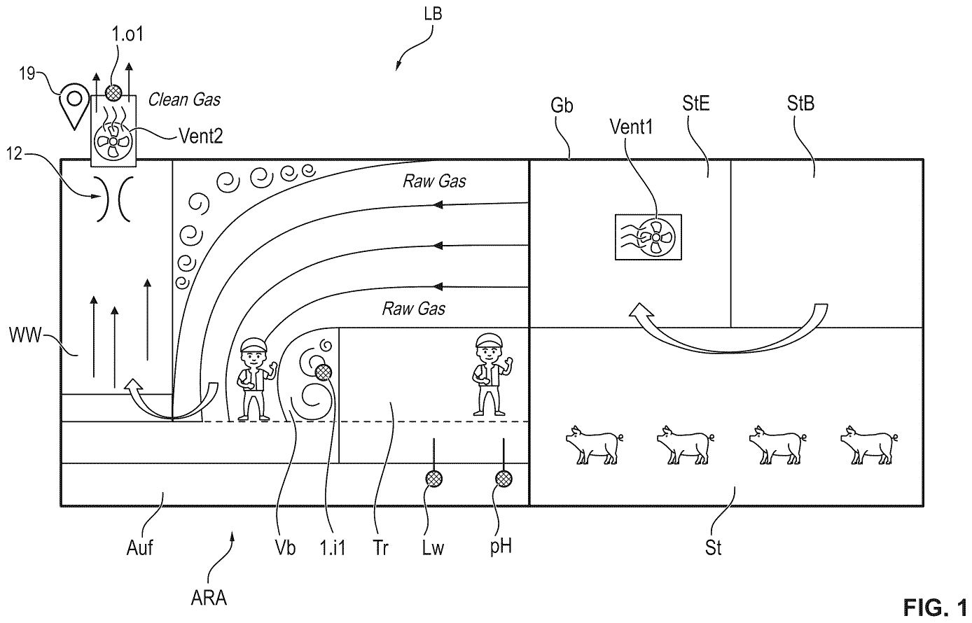

FIG. 1 shows an agricultural enterprise with a chemically operating exhaust gas purification system, a clean gas sensor, a raw gas sensor, an electrical conductivity sensor and a pH sensor according to one or more examples;

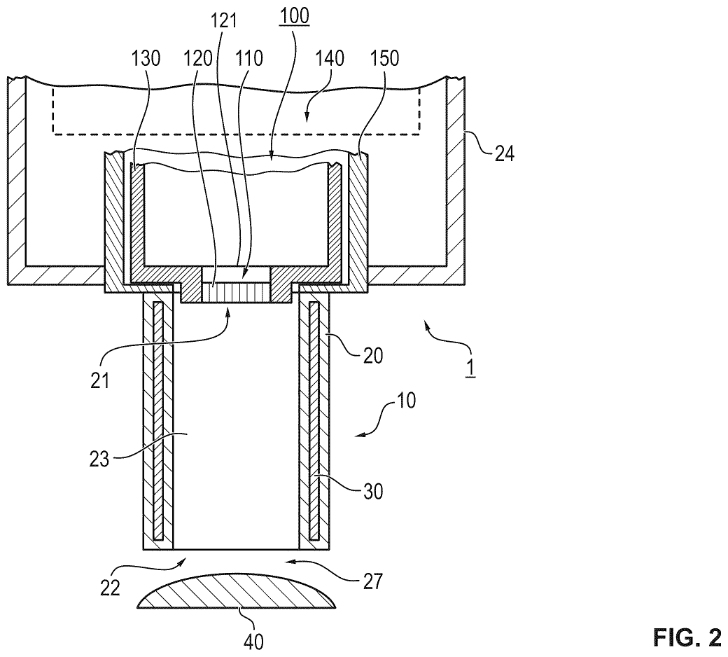

FIG. 2 is a cross-sectional view of a part of an embodiment of the ammonia sensor according to one or more examples;

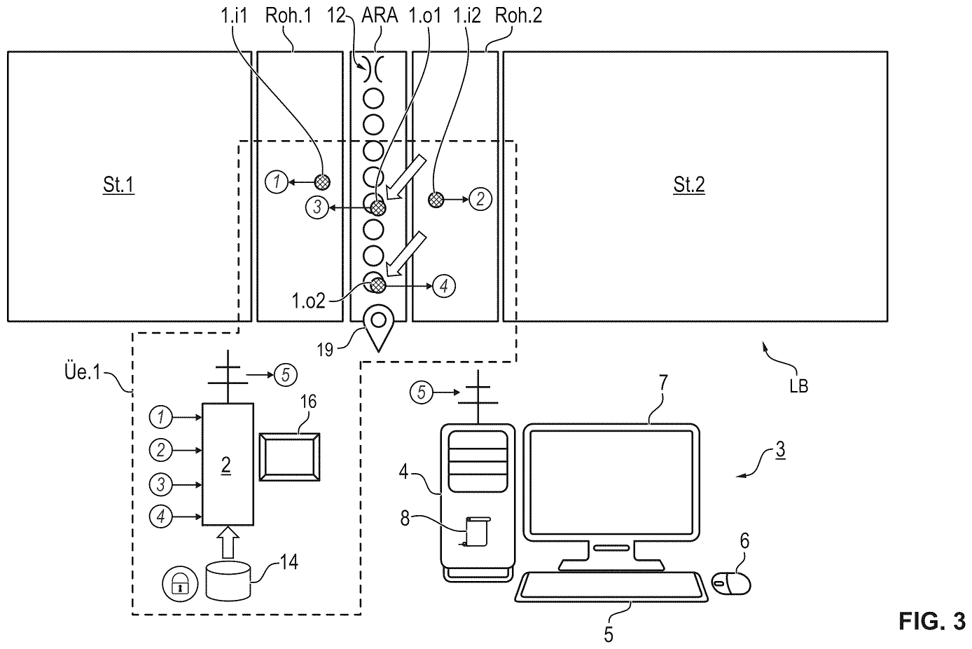

FIG. 3 shows an agricultural enterprise and a monitoring assembly with a monitoring unit and a central computer according to one or more examples;

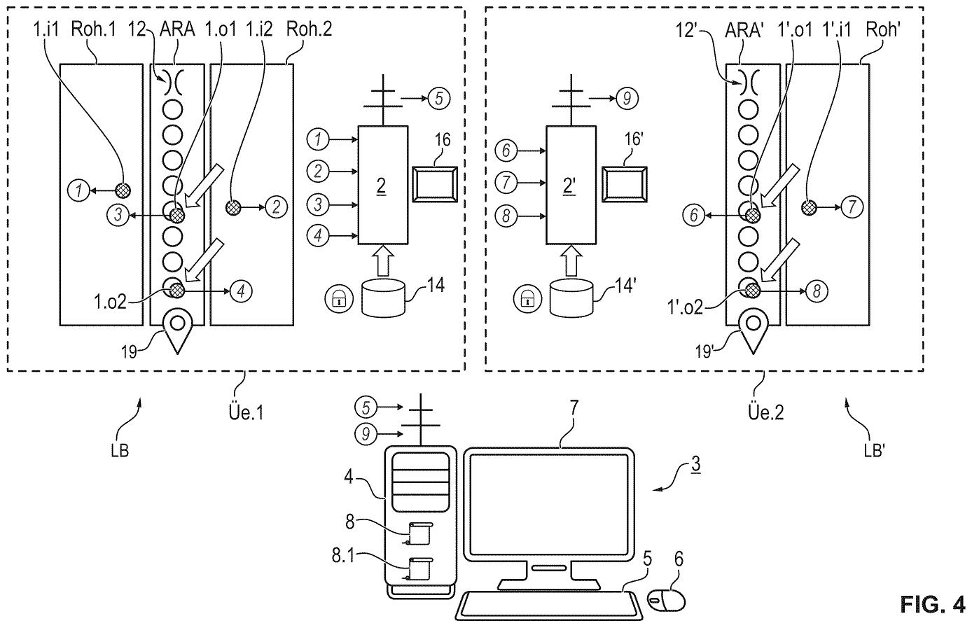

FIG. 4 shows two agricultural enterprises and a monitoring assembly with two monitoring units and the central computer of FIG. 3 according to one or more examples;

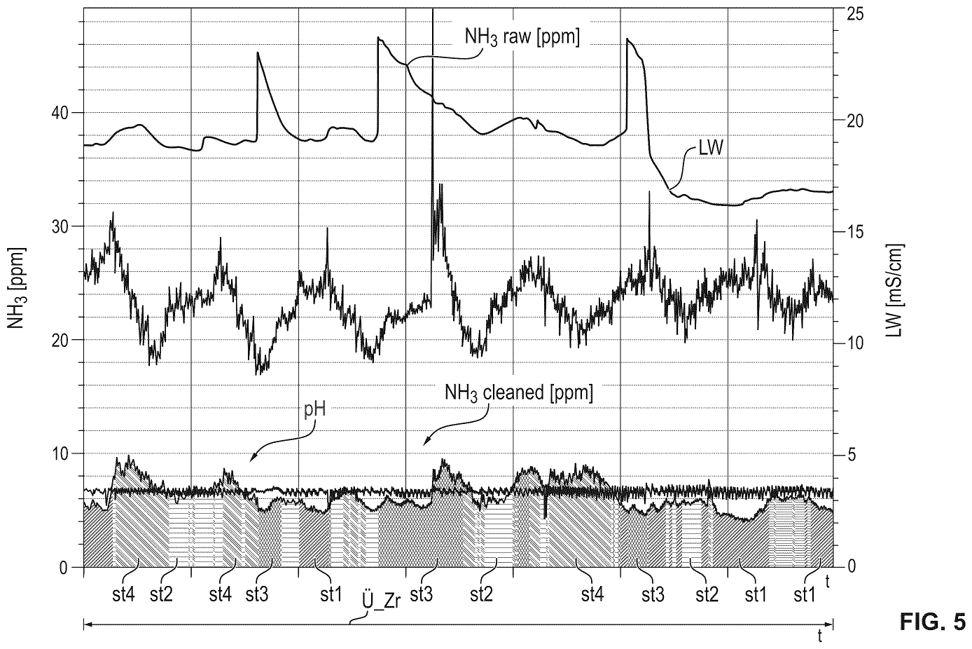

FIG. 5 shows examples of the temporal courses of a plurality of measured physical variables and an evaluation according to one or more examples;

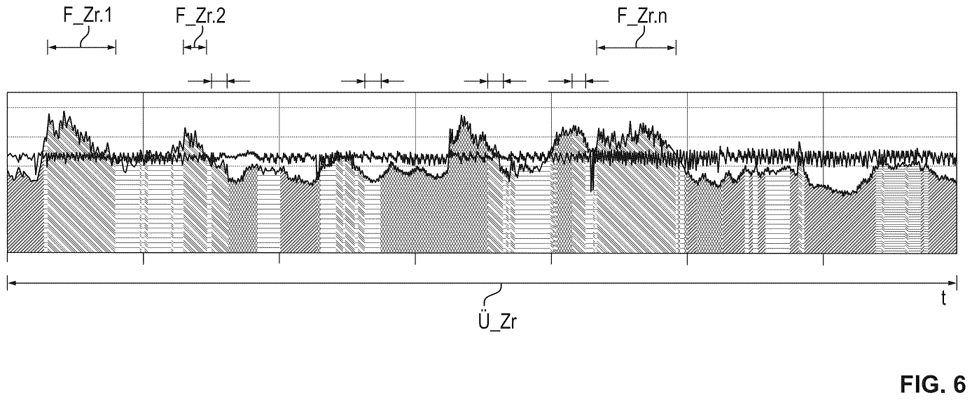

FIG. 6 shows a plurality of error periods in a monitoring period that are obtained by evaluating the curves in FIG. 5, i.e., periods in which too much ammonia is emitted according to one or more examples;

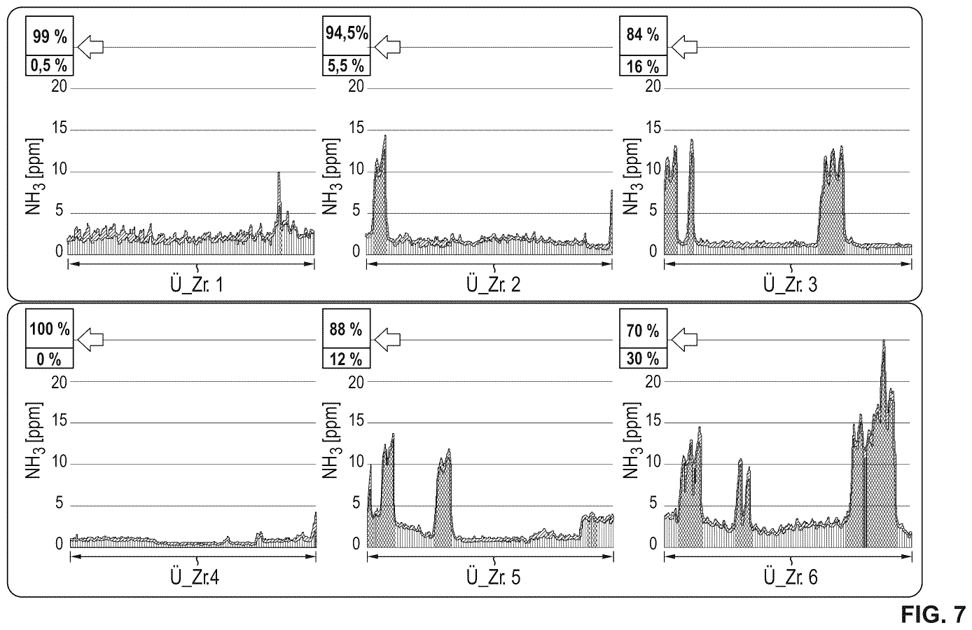

FIG. 7 shows an example of how the respective monitoring result is clearly represented in six periods of time according to one or more examples;

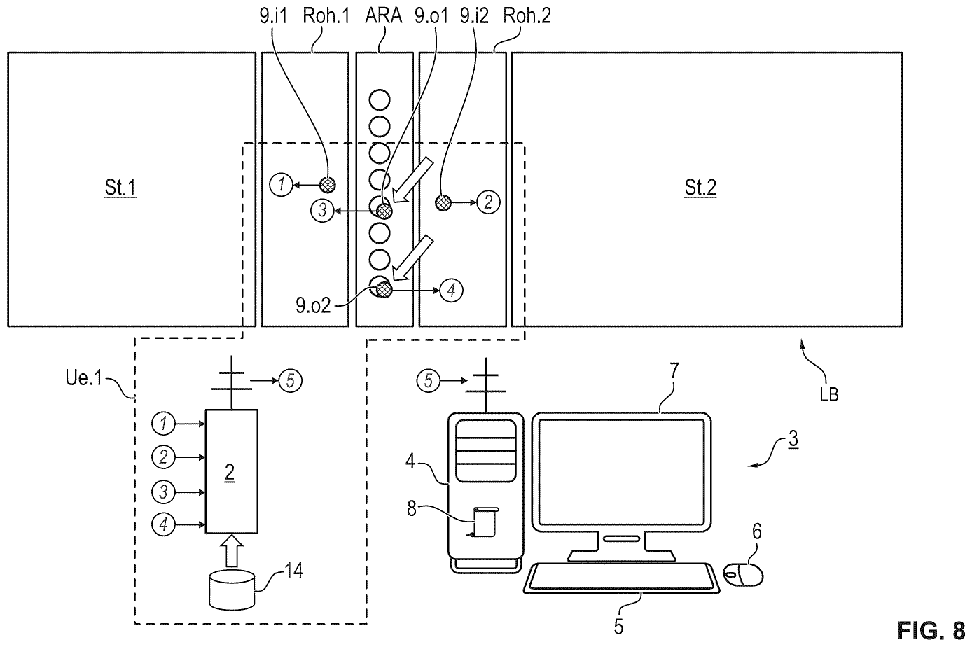

FIG. 8 shows the embodiment of FIG. 3 with signal transmitters instead of ammonia sensors according to one or more examples.

FIG. 1 shows an example of a side view of an agricultural enterprise LB in which the present disclosure can be implemented. A stable St is located in a building Gb. Four pigs are shown schematically in the stable St. The stable St is aerated in a region StB and deaerated in a region StE by means of a first fan Vent1.

The first fan Vent1 conveys a gas mixture from the stable St, the gas mixture usually having a relatively high ammonia content (NH₃). This gas mixture is referred to below as raw gas. The raw gas flows from the region StE almost horizontally over the roof of a technical room Tr and then flows down a wall of the technical room Tr. A swirl region Vb is formed at said wall. Due to this swirl region Vb, the ammonia content varies less in space downstream than upstream at a given time.

A second fan Vent2 on the roof of the building Gb extracts gas from the building Gb upward. This gas contains less ammonia than the raw gas, ideally no ammonia at all, and is called clean gas. A volume (or mass) flow sensor 12 measures the volume flow or mass flow of the clean gas that is extracted from the building Gb upward.

The volume flow is the volume of clean gas that flows into the environment per unit of time. Accordingly, the mass flow is the mass per unit of time. A synonym for volume flow is volumetric flow.

The exhaust gas purification system ARA described below removes ammonia from the raw gas and thereby produces clean gas.

The raw gas flows upward along a scrubber wall WW. A cleaning liquid is applied from above onto the scrubber wall WW and flows down the scrubber wall WW. At the scrubber wall WW, sulfuric acid is added to the raw gas passing by, for example as part of the cleaning liquid. The addition of sulfuric acid (H₂SO₄) causes the chemical reaction H₂SO₄+2 NH₃→(NH₄)₂SO₄. A collection container Auf obliquely below the scrubber wall WW collects the cleaning liquid containing the ammonium sulfate [(NH₄)₂SO₄].

An ammonia sensor 1.i1 measures the ammonia content in the raw gas. An ammonia sensor 1.o1 measures the ammonia content in the clean gas. In the exemplary embodiment, the ammonia sensor 1.i1 for the raw gas is arranged in the swirl region Vb; the ammonia sensor 1.o1 for the clean gas is arranged on the roof and above the second fan Vent2.

An optional schematically shown geoposition sensor 19 measures its own geoposition. This measured geoposition corresponds sufficiently accurately to the geoposition of the clean gas sensor 1.o1.

The product of the ammonia content in the clean gas and the volume flow or mass flow of the clean gas provides the volume flow or mass flow of the emitted ammonia. The integral over the ammonia volume flow or mass flow and over a given reference period provides the volume or mass of ammonia that the agricultural enterprise LB emits in total during the reference period.

A pH sensor pH measures the pH value of the cleaning liquid in the collection container Auf. A conductivity sensor LW measures the electrical conductivity of the cleaning liquid in the collection container Auf. A limitation of an exhaust gas purification system ARA operating as just outlined is as follows: if the electrical conductivity of the cleaning liquid in the collection container Auf is greater than an upper limit or the pH value is lower than a lower limit, the exhaust gas purification system ARA may not be operating correctly. Chemical decomposition or degradation processes may occur in the cleaning liquid in the collection container Auf, and NH₃ can escape as a result thereof. This is undesirable. High electrical conductivity and low pH are two indicators of such undesirable processes. The upper limit for the conductivity is preferably less than 300 mS/cm and is particularly preferably between 5 and 50 mS/cm (mS=milli-Siemens), in particular between 10 and 35 mS/cm. The lower limit for the pH value is preferably between 5 and 8.

In some examples, the cleaning liquid containing the ammonium sulfate is regularly extracted from the collection container Auf. The ammonium sulfate often collects at the bottom of the collection container Auf. In one embodiment, the ammonium sulfate is extracted and disposed of or used, for example, as fertilizer. In another embodiment, at least some of the ammonium sulfate is removed from the cleaning liquid by a chemical process, for example by targeted chemical decomposition. A sufficient amount of water is added to the cleaning liquid and then the cleaning liquid is returned to the scrubber wall WW. Preferably, closed-loop control is used to ensure that the pH value of the cleaning liquid fed to the scrubber wall WW is within a specified range. The pH sensor pH measures the actual pH value of the cleaning liquid after the ammonium sulfate is removed from the cleaning liquid. If necessary, the pH value of the cleaning liquid is increased or decreased. For example, an acid is added to lower the pH value.

An example embodiment of an ammonia sensor 1 is described below with reference to FIG. 2. Both the two raw gas sensors 1.i1, 1.i2 and the two clean gas sensors 1.o1, 1.o2 of FIG. 1, FIG. 3 and FIG. 4 can be constructed as described below. The terms “top” and “bottom” described below relate to an orientation of the ammonia sensor 1 during regular operation.

The ammonia sensor 1 comprises a sensor cell 100 with a measuring chamber. A wall 130 and a porous protective filter 120 surround the sensor cell 100. The sensor cell 100 measures the ammonia content in a gas sample, which is located in the measuring chamber with the wall 130. The sensor cell 100 uses at least one of multiple known measuring principles to measure the ammonia content in a gas mixture.

The gas sample flows from below through an inlet opening 22 into a tubular feed unit 10 and “settles” in a region 23 of the feed unit 10. The settled gas sample flows upward through the feed unit 10 and through an outlet opening 21 into a region 110 of the measuring chamber. This region is delimited by the wall 130, the outlet opening 21, and a membrane 121. Above the membrane 121, an electrolyte and multiple electrodes are located in the measuring chamber. The protective filter 120 is arranged between the feed unit and the region 110 of the measuring chamber.

The feed unit 10 is delimited by a wall 20. A heating element 30 is inserted into the wall 20. The heating element 30 causes a convection flow through the inlet opening 22 and upward into the feed unit 10 (chimney effect). A mechanical impact protection 40 reduces the risk of the convection flow entraining particles into the feed unit 10. An annular inlet gap 27 is formed between the impact protection 40 and the inlet opening 22. Incoming gas is often swirled in said inlet gap 27, which leads to a smaller spatial variation of the ammonia content in the gas sample. The resulting swirl is therefore desirable.

The ammonia sensor 1 further comprises a housing 24, which surrounds an interior region 140. A power supply unit and a dedicated signal-processing sensor evaluation unit are arranged in the interior region 140. By virtue of the dedicated power supply unit, the ammonia sensor 1 is independent of a stationary power supply network. The sensor evaluation unit supplies a signal that comprises information about a measured ammonia concentration and preferably an identifier of the ammonia sensor 1. A holder 150 is inserted in the bottom of the housing 24. Said holder 150 holds the feed unit 10 and surrounds the wall 130 of the sensor cell 100.

FIG. 3 is a schematic diagram of an exhaust gas purification system ARA in an agricultural enterprise LB according to one or more examples. Animals are kept in two stables St.1, St.2 of the agricultural enterprise LB. Therefore, ammonia can be produced in each stable St.1, St.2. In a region called Roh.1, raw gas escaping from the stable St.1 can accumulate. Accordingly, raw gas escaping from the stable St.2 can accumulate in a Roh.2 region.

The exhaust gas purification system ARA purifies raw gas coming from one of the stables St. 1, St.2. At least one upper limit value is predetermined for this agricultural enterprise LB. The or each predetermined upper limit value specifies the maximum amount of ammonia that this agricultural enterprise LB is permitted to emit. For example, an upper limit value for the ammonia concentration in an emitted gas mixture and/or an upper limit value for the maximum amount of ammonia that the agricultural enterprise LB is permitted to emit in a given reference time period are predetermined. The agricultural enterprise LB emits the clean gas that leaves the exhaust gas purification system ARA. The volume flow sensor 12 measures the volume flow or mass flow of the clean gas.

A first monitoring unit Ue.1, which is described below, is assigned to this exhaust gas purification system ARA. The exhaust gas purification system ARA is configured to remove ammonia from both the raw gas of the Roh.1 region and the raw gas of the Roh.2 region. The exhaust gas purification system ARA can be configured as described with reference to FIG. 1.

A first raw gas sensor 1.i1 is arranged in the region Roh.1 and is configured to measure the ammonia content in the gas mixture present in the region Roh.1. Accordingly, a second raw gas sensor 1.i2 is arranged in the region Roh.2 and is configured to measure the ammonia content in the gas mixture present in the region Roh.2. The two raw gas sensors 1.i1, 1.i2 measure the ammonia content at two different measuring positions, and the ammonia content can vary from measuring position to measuring position.

Two clean gas sensors 1.o1, 1.o2 are arranged downstream of the exhaust gas purification system ARA. These clean gas sensors 1.o1, 1.o2 measure the ammonia content in clean gas at two spaced-apart measuring positions and each act as a first ammonia sensor.

Each sensor 1.i1, 1.i2, 1.o1, 1.o2 generates a respective signal, which contains information about the ammonia content measured by the sensor 1.i1, 1.i2, 1.o1, 1.o2. The ammonia content is preferably stated in ppm (parts per million), alternatively in vol. % or wt. %. The sensors 1.i1, 1.i2, 1.o1, 1.o2 transmit their signals to a communication unit 2, which acts as the first communication unit. In one embodiment, the communication unit 2 regularly queries the sensors 1.i1, 1.i2, 1.o1, 1.o2, and in response to a query, the queried sensor 1.i1, 1.i2, 1.o1, 1.o2 transmits at least one signal value. The communication unit 2 is also arranged in the agricultural enterprise LB. The signals are transmitted via cable and/or via radio waves from the sensors 1.i1, 1.i2, 1.o1, 1.o2 to the communication unit 2.

The ammonia sensors 1.i1, 1.i2, 1.o1, 1.o2, the volume flow sensor 12, the geoposition sensor 19, and the communication unit 2 belong to the first monitoring unit Ue.1, which is assigned to the agricultural enterprise LB having the exhaust gas purification system ARA.

FIG. 4 shows two agricultural enterprises LB and LB′ according to one or more examples. The agricultural enterprise LB comprises the exhaust gas purification system ARA and the first monitoring unit Ue.1 of FIG. 1. The agricultural enterprise LB′ comprises a further exhaust gas purification system ARA′ and a second monitoring unit Ue.2. The further exhaust gas purification system ARA′ removes ammonia from raw gas, which can accumulate in a region Roh′. The second monitoring unit Ue.2 comprises a further raw gas sensor 1′.i1, two further clean gas sensors 1′.o1, 1′.o2 and a further communication unit 2′. A respective data connection between a further sensor 1′.i1, 1′.o1, 1′.o2 and the second communication unit 2′ is established at least temporarily. In addition, a data connection is established at least temporarily between the second communication unit 2′ and the central computer 3, the central computer 3 already having been described with reference to FIG. 3.

An optional schematically shown geoposition sensor 19, 19′ measures its own geoposition, and this measured geoposition corresponds sufficiently accurately to the geoposition of a clean gas sensor 1.o1, 1.o2 or 1′.o1, 1′.o2 of the monitoring unit Ue.1 or Ue.2. An optional schematically shown volume flow sensor 12, 12′ measures the volume flow or mass flow of the clean gas from the particular exhaust gas purification system ARA or ARA′.

In the example shown in FIG. 4, the central computer 3 receives signals from the first communication unit 2 as well as signals from the second communication unit 2′. Each communication unit 2, 2′ belongs to a monitoring unit Ue.1, Ue.2.

The data memory 14 stores an upper limit value for the maximum amount of ammonia that the agricultural enterprise LB is permitted to emit. Accordingly, an upper limit value for the maximum amount of ammonia that the agricultural enterprise LB′ is permitted to emit is stored in a data memory 14′ of the second monitoring unit Ue.2. As mentioned above, an upper limit value can in particular specify a maximum permissible instantaneous ammonia concentration or the maximum permissible amount of ammonia in a reference time period.

Each upper limit value stored in a data memory 14, 14′ is protected against external changes, in particular against intentional manipulation. This is indicated by corresponding symbols. In particular, each stored upper limit value is preferably encrypted. However, it is possible for an authorized person with appropriate access authorization to overwrite a stored upper limit value with a new upper limit value, so that a new upper limit value is stored in the data memory 14, 14′ and subsequently used. Preferably, a list of information about authorized persons is stored in each data memory 14, 14′, the information comprising access information, for example a password and/or a QR code and/or biometric features.

The communication unit 2, 2′ has, at least temporarily, read access to the data memory 14, 14′. The communication unit 2, 2′ and the data memory 14, 14′ belong to the particular monitoring unit for the agricultural enterprise LB, LB′. In addition to the upper limit value for the agricultural enterprise LB, LB′ mentioned above, the following information is stored in this data memory 14, 14′:
a unique identifier of the monitored agricultural enterprise LB, LB′, which identifier distinguishes this agricultural enterprise LB, LB′ from any other monitored agricultural enterprise,
which cleaning principle the monitored exhaust gas purification system ARA, ARA′ uses, for example whether it removes ammonia from the raw gas biologically or chemically or in both ways,
optionally, a predefined identifier of a geoposition region of the monitored agricultural enterprise LB, LB′, each location within this agricultural enterprise LB, LB′ having a geoposition that lies within this geoposition region,
a unique identifier for each sensor 1.i1, 1.i2, 1.o1, 1.o2, 1′.i1, 1′.o1, 1′.o2, 19, 19′, 12, 12′, the unique identifier distinguishing this sensor 1.i1, 1.i2, 1.o1, 1.o2, 1′.i1, 1′.o1, 1′.o2 from any other sensor in the same enterprise LB, LB′,
in the case of an ammonia sensor, optionally, whether this sensor is located upstream or downstream of the monitored exhaust gas purification system ARA, ARA′, i.e., whether it measures raw gas or clean gas, and
a unique identifier for each of the sensors 19, 19′, 12, 12′.

FIG. 3 shows an optional output unit 16 of the first monitoring unit Ue.1, and FIG. 4 shows two optional output units 16, 16′. On this output unit 16, 16′, a stored upper limit value valid for the particular enterprise LB, LB′ as well as measured values for the actually emitted amount or concentration of ammonia are displayed. Preferably, the upper limit value and the displayed measured values have the same unit of measurement. If necessary, measured values from the ammonia sensors are converted into the output unit of measurement. Preferably, a warning or alarm is output on the output unit 16, 16′ if the actual emissions exceed an upper limit value or are likely to exceed it according to a temporal extrapolation.

A central computer 3 is located at a distance from the monitored agricultural enterprise LB, LB′ and therefore also at a distance from the exhaust gas purification system ARA, ARA′ and the monitoring unit Ue.1, Ue.2. In the exemplary embodiment, the central computer 3 comprises the following components:
a signal-processing computing unit 4,
a screen 7,
a keyboard 5, and
a mouse 6.

The communication unit 2 generates a message. This message comprises the signals and/or measured values of sensors 1.i1, 1.i2, 1.o1, 1.o2, 12, 19. The communication unit 2 causes this message to be transmitted to the central computer 3 via cable and/or radio waves. Preferably, the message is transmitted using a public data transmission network and/or mobile network. Each transmitted signal is provided with the above-described unique identifier of a sensor 1.i1, 1.i2, 1.o1, 1.o2, 12, 19, the signal originating from this sensor. The communication unit 2 determines this unique identifier by having read access to the data memory 14. The same applies to the communication unit 2′.

The computing unit 4 comprises a processor and a data memory. At least one evaluation program 8, 8.1 is stored in the data memory. The processor is configured to execute the evaluation program 8, 8.1 and of processing received signals during execution. The central computer 3 received these signals from the first monitoring unit Ue.1 and optionally from a second monitoring unit Ue.2 and at least one further monitoring unit. In one embodiment, the evaluation program 8 relates to biologically operating exhaust gas purification systems, while the evaluation program 8.1 relates to chemically operating ones.

In one or more examples, each communication unit 2, 2′ of a monitoring unit Ue.1, Ue.2 transmits a message to the central computer 3 at regular intervals, for example with a fixed sampling rate. This message comprises:
the stored unique identifier of the agricultural enterprise LB, LB′, the monitoring unit Ue.1, Ue.2 being assigned to this agricultural enterprise LB, LB′,
optionally, the stored geoposition region of the agricultural enterprise LB, LB′,
optionally, the geoposition measured by a geoposition sensor 19, 19′, which corresponds to the geoposition of a clean gas sensor,
optionally, the volume flow or mass flow measured by a volume flow sensor 12, 12′,
a unique identifier for each sensor 1.i1, 1.i2, 1.o1, 1.o2, 1′.i1, 1′.o1, 1′.o2, 12, 12′, 19, 19′ of the monitoring unit Ue.1, Ue.2,
for each ammonia sensor 1.i1, 1.i2, 1.o1, 1.o2, 1′.i1, 1′.o1, 1′.o2 of the monitoring unit Ue.1, Ue.2, an indicator or a characterization of the ammonia concentration or the amount of ammonia that this sensor 1.i1, 1.i2, 1.o1, 1.o2, 1′.i1, 1′.o1, 1′.o2 is currently measuring,
optionally, the measured electrical conductivity LW and the measured pH value of a liquid used in a biologically operating exhaust gas purification system, see FIG. 1,
whether the monitored exhaust gas purification system ARA, ARA′ operates biologically or chemically or in both ways, and
the or each upper limit value stored in the data memory 14, 14′ for this agricultural enterprise LB, LB′, the upper limit value specifying a maximum permissible ammonia emission.

The processor of the computing unit 4 executes the or at least one evaluation program 8, 8.1. The executed evaluation program 8, 8.1 evaluates the messages from each connected communication unit 2, 2′. Through the evaluation, the evaluation program 8, 8.1 determines the following information:
From which agricultural enterprise LB, LB′ does the message originate?
According to the message, what is the stored geoposition region of this agricultural enterprise LB, LB′?
According to the message, what is the current upper limit value for ammonia emissions for this agricultural enterprise LB, LB′?
Which sensor 1.i1, 1.i2, 1.o1, 1.o2, 1′.i1, 1′.o1, 1′.o2 of the monitoring unit Ue.1, Ue.2 measured which ammonia content? And is this sensor 1.i1, 1.i2, 1.o1, 1.o2, 1′.i1, 1′.o1, 1′.o2 located upstream or downstream of the particular exhaust gas purification system ARA, ARA′?
optionally, a measured geoposition of a clean gas sensor,
optionally, the measured volume flow or mass flow,
How does the monitored exhaust gas purification system ARA, ARA′ operate?
Optional: What electrical conductivity and what pH value were measured for this exhaust gas purification system ARA, ARA′?

It is possible that a monitoring unit Ue.1, Ue.2 comprises at least two spaced-apart sensors 1.i1, 1.i2, 1.o1, 1.o2, 1′.o1, 1′.o2 for the ammonia content in the raw gas and/or for the ammonia content in the clean gas; see FIG. 3 and FIG. 4. In this case, the evaluation program 8, 8.1 averages the measured ammonia concentration in the raw gas and/or in the clean gas.

It is possible that a raw gas sensor and/or a clean gas sensor may work incorrectly or may have even failed completely. Usually, a faulty sensor will provide an ammonia content that is too low, a failed sensor will even provide an ammonia content of 0, but not an ammonia content that is too high.

The evaluation program 8, 8.1 on the computing unit 4 or an evaluation program of the monitoring unit Ue.1, Ue.2 can in some cases automatically detect an error and a failure of a sensor and can compensate to a certain extent. The following events indicate a sensor fault:
The or a clean gas sensor of a monitoring unit Ue.1, Ue.2 provides a higher ammonia content than the or a raw gas sensor of this monitoring unit Ue.1, Ue.2, and there is a time span between the two sampling times of these two sensors that is smaller than a predetermined lower limit. The deviation between the two measured ammonia values is also greater than a predetermined upper limit. In practice, the ammonia content cannot vary to such an extent. As a general rule, the raw gas sensor providing the lower ammonia content is then defective or faulty.

The time span is taken into account because the actual ammonia content may not have changed to such an extent during this time span, but over a longer period the ammonia content in the raw gas may actually decrease.
The temporal course of the ammonia concentration in the clean gas and/or in the raw gas decreases faster than a specified change limit. This is often an indication that the sensor that measured this ammonia concentration has become defective. The change limit is chosen such that a decrease that is faster than the change limit cannot correspond to the actual course of an ammonia concentration.
A monitoring unit Ue.1, Ue.2 comprises two raw gas sensors. At one sampling time, the absolute or percentage deviation between the two measured values, i.e., between the two measured ammonia contents, of these two raw gas sensors is greater than a specified lower limit. Or the absolute or percentage deviation increases more than a specified lower limit.

This is an indication that the raw gas sensor that provides the lower ammonia content is working incorrectly or has even failed completely.
The same applies to a monitoring unit Ue.1, Ue.2 with two clean gas sensors.

The evaluation program 8 responds as follows to the detection that an ammonia sensor is faulty:
If a monitoring unit Ue.1, Ue.2 has only a single raw gas sensor and said raw gas sensor has failed, the evaluation program 8 generates an error message. The same applies if a monitoring unit Ue.1, Ue.2 has only a single clean gas sensor and said sensor has failed.
If a monitoring unit Ue.1, Ue.2 has two raw gas sensors and one of them measures incorrectly or has failed, the evaluation program 8 uses the measured value of the sensor that measures a higher ammonia content. The same applies to a monitoring unit Ue.1, Ue.2 with two clean gas sensors. As a rule, an error or failure of a sensor usually leads to a measured value of the ammonia content that is too low, but not to a measured value that is too high.
If a monitoring unit Ue.1, Ue.2 comprises two raw gas sensors and these two sensors are intact, the evaluation program 8, 8.1 determines the ammonia content in the raw gas by averaging, e.g., averaging in a weighted manner, the measured values of these two raw gas sensors. The same applies to a monitoring unit with two clean gas sensors.

In one or more examples, the evaluation program 8, 8.1 performs a plausibility check and uses various geopositions for this purpose. As explained above, on the one hand, a geoposition region of the monitored agricultural enterprise LB, LB′ is transmitted as part of a message, this geoposition region being stored in the data memory 14, 14′. On the other hand, a geoposition measured by a geoposition sensor 19, 19′ is transmitted as part of the message. The measured geoposition matches the geoposition of a clean gas sensor 1.o2, 1′.o2. The following events indicate an error:
The measured geoposition of the clean gas sensor 1.o2, 1′.o2 lies outside the geoposition region of the agricultural enterprise LB, LB′ by more than a specified tolerance.
According to the transmitted identifier, two different messages originate from the same agricultural enterprise LB, LB′. However, the two transmitted geoposition regions differ from each other by more than a specified tolerance.
Two different messages originate from two different agricultural enterprises LB, LB′ according to the particular transmitted identifier and/or according to the transmitted geoposition region. However, the two measured geopositions, each transmitted as part of these two messages, correspond to one another to within a specified tolerance.

The following describes an example of an evaluation carried out by the evaluation program 8, 8.1.

The central computer 3 captures a user input made by a user using the keyboard 5 and/or the mouse 6. Said user input specifies which evaluations the central computer 3 should generate and display. In addition, the user specification specifies a monitoring period U_Zr to which the or each generated representation is to relate. The central computer 3 captures the user input and generates at least one representation according to the captured user input. The central computer 3 causes the desired representation to be output on the screen 7 in a form that can be perceived by a human.

As already explained, each message from a communication unit 2, 2′ comprises the measured current ammonia concentration and the measured volume flow or mass flow of the clean gas. Using these two measured values, the evaluation program 8, 8.1 determines which volume or amount of ammonia flows into the environment per unit of time together with the clean gas. For example, the evaluation program 8, 8.1 multiplies the measured ammonia concentration by the measured volume flow or mass flow. The result of the determinations is abbreviated below as the current ammonia content.

FIG. 5 shows the exemplary temporal course of various physical variables in the agricultural enterprise LB, these physical variables having been measured as described above by sensors of the monitoring unit Ue.1 and having been transmitted as part of at least one message from the communication unit 2 to the central computer 3. Time is plotted on the x-axis. The current ammonia content NH₃ in [ppm] is plotted on the y-axis on the left, and the electrical conductivity LW in [mS/cm] is plotted on the right. In FIG. 5:
NH₃ raw means the current ammonia content in [ppm] in the raw gas, i.e., before exhaust gas purification,
NH₃ cleaned means the current ammonia content in [ppm] in the clean gas, i.e., after exhaust gas purification,
LW means the value for the electrical conductivity in [mS/cm] of the liquid in which ammonium sulfate is produced during cleaning, and
pH means the pH value of this liquid.

The evaluation program 8, 8.1 compares, in particular, the current ammonia content in the clean gas with the or each transmitted upper limit value and checks whether the transmitted electrical conductivity and the transmitted pH value are within the permissible range or not. The agricultural enterprise LB has one of the following four states at any given time:
st1: Green range: The current ammonia content is well below the upper limit value, and the electrical conductivity and pH value are within the permissible range.
st2: Yellow range: The current ammonia content is only slightly below the upper limit value.
st3: The current ammonia content is well below the upper limit value, but the electrical conductivity and/or pH value lie outside of the permissible range, such that the measurement results may be erroneous because ammonia can be released due to decomposition of the cleaning liquid,
st4: Red range: The current ammonia content is above the upper limit value.

The representation in FIG. 6 shows each detected error period F_Zr.1, F_Zr.2, . . . in the specified monitoring period U_Zr. An error period is a period of a specified minimum length during which the current ammonia content in the clean gas is continuously above the predetermined upper limit value. As already explained, this upper limit value can vary for different agricultural enterprises LB, LB′ and can also be dependent on the season, the time of day and/or the type of operation of the enterprise LB, LB′.

In one or more examples, the evaluation program 8, 8.1 additionally determines the total volume or amount of ammonia that has been released into the environment of the agricultural enterprise LB, LB′ since the beginning of the monitoring period U_Zr. For this purpose, the evaluation program 8, 8.1 performs a numerical integration over the monitoring period U_Zr.

FIG. 7 shows an example of how the particular monitoring result is clearly represented in six periods of time. The particular evaluation for six monitoring periods U_Zr.1, . . . , U_Zr.6 is shown in a single representation. The upper percentage indicates how long the total time periods in the particular monitoring period U_Zr.1, . . . , U_Zr.6 are in which the ammonia emissions are below the upper limit value. The lower percentage indicates the total duration of the error periods in the particular monitoring period U_Zr.1, . . . , U_Zr.6.

In the following, an embodiment is described with reference to FIG. 8 in which, in particular, the data connections of the monitoring assembly according to the present disclosure can be checked. The following description refers to the embodiment according to FIG. 3, in which two raw gas sensors 1.i1, 1.i2 and two clean gas sensors 1.o1, 1.o2 are used to check the exhaust gas purification system ARA. Same reference signs have the same meanings as in FIG. 3.

The monitoring assembly can be operated either in a monitoring mode or in a checking mode according to the embodiment described below. In monitoring mode, each ammonia sensor 1.i1, 1.i2, 1.o1, 1.o2 generates and supplies a signal. This signal comprises information about the measured ammonia content. The evaluation program 8 determines the particular ammonia content in the raw gas and in the clean gas for the exhaust gas purification system ARA and optionally for each additional monitored exhaust gas purification system ARA′, specifically for each sampling time, as described above.

The following describes how the monitoring assembly is operated in checking mode. In checking mode, each ammonia sensor 1.i1, 1.i2, 1.o1, 1.o2 is replaced by one signal transmitter 9.i1, 9.i2, 9.o1, 9.o2. It is preferable to ensure in advance that each signal transmitter 9.i1, 9.i2, 9.o1, 9.o2 is operating correctly. The terms “raw gas signal transmitter” 9.i1, 9.i2 and “clean gas signal transmitter” 9.o1, 9.o2 are used below.

In one or more examples, each ammonia sensor 1.i1, 1.i2, 1.o1, 1.o2 is physically replaced by one signal transmitter 9.i1, 9.i2, 9.o1, 9.o2. In another embodiment, the first monitoring unit Ue.1 permanently comprises each ammonia sensor 1.i1, 1.i2, 1.o1, 1.o2 as well as each signal transmitter 9.i1, 9.i2, 9.o1, 9.o2. In monitoring mode, the ammonia sensors 1.i1, 1.i2, 1.o1, 1.o2 are activated and the signal transmitters 9.i1, 9.i2, 9.o1, 9.o2 are deactivated. Conversely, when operating in checking mode, the ammonia sensors are deactivated and the signal transmitters 9.i1, 9.i2, 9.o1, 9.o2 are activated. In one embodiment, each signal transmitter 9.i1, 9.i2, 9.o1, 9.o2 is a component of an ammonia sensor 1.i1, 1.i2, 1.o1, 1.o2, for example an electronic circuit, and can be activated and deactivated.

Each signal transmitter 9.i1, 9.i2, 9.o1, 9.o2 preferably comprises a dedicated power supply unit and also supplies a signal that comprises information about an ammonia content, the signal being an electrical signal in one embodiment and a digital signal in another embodiment that has the same data format as the signal of an ammonia sensor 1.i1, 1.i2, 1.o1, 1.o2.

However, this ammonia content in the generated signal was not measured, but was specified to the signal transmitter by a user or by the evaluation program 8 or by another unit. The signal provided by the signal transmitter therefore comprises information about an ammonia content specified to the signal transmitter. Preferably, each signal transmitter 9.i1, 9.i2, 9.o1, 9.o2 comprises an input unit with which a user can specify an ammonia content. It is also possible for each signal transmitter 9.i1, 9.i2, 9.o1, 9.o2 to be controlled remotely, for example by the evaluation program 8, and for the signal transmitter 9.i1, 9.i2, 9.o1, 9.o2 to be given an ammonia content by means of this control. Each signal transmitter 9.i1, 9.i2, 9.o1, 9.o2 thus replaces and emulates an ammonia sensor 1.i1, 1.i2, 1.o1, 1.o2.

As explained above, when operating in monitoring mode, the evaluation program 8 determines the particular ammonia content in the raw gas and in the clean gas for each sampling time. For this purpose, the evaluation program 8 uses received signals from the ammonia sensors 1.i1, 1.i2, 1.o1, 1.o2. In addition, the evaluation program 8 calculates a quality function in the form of a degree of cleaning Rg=Rg(Amm_{clean}, Amm_{raw}) for each sampling time. Preferably, Rg is a dimensionless number between 0 and 1. Rg=1 means that the raw gas comprises ammonia, but the clean gas does not comprise ammonia at all.

In the checking mode, an ammonia content in the raw gas is specified to each raw gas signal transmitter 9.i1, 9.i2. If there are a plurality of raw gas signal transmitters, in one embodiment each raw gas signal transmitter 9.i1, 9.i2 is assigned the same ammonia content Amm_{raw} in the raw gas, in another embodiment two different values for the ammonia content, for example Amm_{raw}+Δ or Amm_{raw}−Δ. This also makes it possible to check whether the evaluation program 8 correctly aggregates measured values into a single value. The same applies to the clean gas signal transmitters 9.o1, 9.o2.

The signals with the respectively specified values for the ammonia content are transmitted by the signal transmitters 9.i1, 9.i2, 9.o1, 9.o2 to the central computer 3 and evaluated by the evaluation program 8. Even when operating in checking mode, the evaluation program 8 determines an ammonia content. The evaluation program 8 uses the received signals from the signal transmitters 9.i1, 9.2, 9.o1, 9.o2 for this determination.

The evaluation program 8 determines a value for the ammonia content in the raw gas, for which the evaluation program 8 uses the signals from the raw gas signal transmitters 9.i1, 9.i2. Accordingly, the evaluation program 8 determines a value for the ammonia content in the clean gas, for which the evaluation program 8 uses the signals from the clean gas signal transmitters 9.o1, 9.o2. In one embodiment, the evaluation program 8 “knows,” for each signal transmitter 9.i1, 9.i2, 9.o1, 9.o2, which value for an ammonia content has been specified to that signal transmitter. The evaluation program 8 compares the values determined for the ammonia content, which were determined by evaluating the signals of the signal transmitters 9.i1, 9.i2, 9.o1, 9.o2, with the corresponding predetermined values. In another embodiment, a user performs this comparison.

In one or more examples, it is checked whether the determined value for an ammonia content deviates from the corresponding predetermined value by more than a specified tolerance. Possible causes for a deviation greater than a specified tolerance include, in particular:
A component of the monitoring assembly is not supplied with electrical voltage at all or not sufficiently. This applies both to a component that has its own power supply unit and to a component that is at least temporarily connected to a stationary power supply network.
A data connection from a signal transmitter 9.i1, 9.i2, 9.o1, 9.o2 to the central computer 3 is interrupted or defective in another way.
Signal processing on the way from a signal transmitter 9.i1, 9.i2, 9.o1, 9.o2 to the evaluation program 8 provides incorrect values.

In one or more examples, each signal transmitter 9.i1, 9.i2, 9.o1, 9.o2 is given a temporal course of an ammonia content. Preferably, the respectively specified value for the ammonia content is varied in such a way that it assumes the value zero at least once and the highest possible value for the ammonia content at least once. In this manner, in particular, the signal processing for the entire possible value range of ammonia content is checked, specifically for both the raw gas and the clean gas. By using temporal courses, a significant delay in data transmission can be detected more easily than if only a single value were specified.

In some examples, the evaluation program 8 can automatically detect whether ammonia sensors 1.i1, 1.i2, 1.o1, 1.o2 or signal transmitters 9.i1, 9.i2, 9.o1, 9.o2 are currently being used, i.e., whether the monitoring assembly is currently being operated in monitoring mode or in checking mode. For example, each signal from a signal transmitter 9.i1, 9.i2, 9.o1, 9.o2, in addition to the specified ammonia content, respectively comprises corresponding information, for example an identifier of the signal transmitter 9.i1, 9.i2, 9.o1, 9.o2 or information indicating that the ammonia content in the transmitted signal has been specified and not measured. It is also possible to specify a test pattern to each signal transmitter 9.i1, 9.i2, 9.o1, 9.o2 as a temporal course of the ammonia content, which test pattern does not occur during actual operation of the monitored exhaust gas purification system ARA.

List of reference signs

| | |
|---|---|
| 1 | Ammonia sensor |
| 1.i1, | Raw gas sensors of the first monitoring unit Ue.1, arranged |
| 1.i2 | upstream of the exhaust gas purification system ARA |
| 1′.i1 | Raw gas sensor of the second monitoring unit Ue.2, |
| | arranged upstream of the exhaust gas purification system |
| | ARA′ |
| 1.o1, | Clean gas sensors of the first monitoring unit Ue.1, arranged |
| 1.o2 | downstream of the exhaust gas purification system ARA |
| 1′.o1, | Clean gas sensors of the second monitoring unit Ue.2, |
| 1′.o2 | arranged downstream of the exhaust gas purification system |
| | ARA′ |
| 2 | First communication unit, receives signals from the raw gas |
| | sensors 1.i1, 1.i2, from the clean gas sensors 1.o1, 1.o2, |
| | from the geoposition sensor 19 and from the volume flow |
| | sensor 12, generates a unique identifier for each signal and |
| | transmits the signals with the identifiers to the central |
| | computer 3, belongs to the first monitoring unit Ue.1 |
| 2′ | Second communication unit, receives signals from the raw |
| | gas sensors 1′.i1, from the clean gas sensors 1′.o1, 1′.o2, |
| | from the geoposition sensor 19′ and from the volume flow |
| | sensor 12′, generates a unique identifier for each signal and |
| | transmits the signals with the identifiers to the central |
| | computer 3, belongs to the second monitoring unit Ue.2 |
| 3 | Central computer 3, comprises the computing unit 4, the |
| | screen 7, the keyboard 5 and the mouse 6, receives signals |
| | from the communication units 2, 2′ |
| 4 | Computing unit of the central computer 3 |
| 5 | Keyboard of the central computer 3 |
| 6 | Mouse of the central computer 3 |
| 7 | Screen of the central computer 3 |
| 8 | Evaluation program on the central computer 3, evaluates the |
| | signals from the sensors 1.i1, 1.i2, 1.o1, 1.o2, acts as the |
| | evaluation unit |
| 8.1 | Further evaluation program on the central computer 3 |
| 9.i1, | Raw gas signal transmitters that replace the raw gas |
| 9.i2 | sensors 1.i1, 1.i2 when operating in checking mode |
| 9.o1, | Clean gas signal transmitters that replace the clean gas |
| 9.o2 | sensors 1.o1, 1.o2 when operating in checking mode |
| 10 | Feed unit, comprises the inlet opening 22 and the outlet |
| | opening 21 |
| 12, | Volume flow sensor, measures the volume flow or mass flow |
| 12′ | of the clean gas into the environment |
| 14 | Data memory in which the unique identifiers of the sensors |
| | 1.i1, 1.i2, 1.o1, 1.o2, 12, 19 and the unique identifier of the |
| | monitoring unit Ue.1 are stored and to which the |
| | communication unit 2 has at least temporary read access |
| 14′ | Data memory in which the unique identifiers of the sensors |
| | 1′.i1, 1′.o1, 1′.o2, 12′, 19′ and the unique identifier of the |
| | second monitoring unit Ue.2 are stored and to which the |
| | second communication unit 2′ has at least temporary read |
| | access |
| 16 | Output unit, belongs to the first monitoring unit Ue.1 |
| 16′ | Output unit, belongs to the second monitoring unit Ue.2 |
| 19, | Geoposition sensor, measures the geoposition of a clean |
| 19′ | gas sensor 1.o2, 1′.o2 |
| 20 | Wall of the feed unit 10 |
| 21 | Outlet opening from the feed unit 10 |
| 22 | Inlet opening into the feed unit 10 |
| 23 | Region in the feed unit 10 in which the gas sample settles |
| 24 | Housing of the ammonia sensor 1, surrounds the interior |
| | region 140 with the power supply unit |
| 27 | Inlet gap between the feed unit 10 and the impact protection |
| | 40 |
| 30 | Heating element in the wall 20 |
| 40 | Mechanical impact protection below the feed unit 10 |
| 100 | Sensor cell with the measuring chamber, measures the |
| | ammonia content |
| 110 | Region of the measuring chamber which receives a gas |
| | sample flowing in from below, is delimited by the wall 130, |
| | the outlet opening 21 and the membrane 121 and belongs to |
| | the sensor cell 100 |
| 120 | Protective filter in front of the sensor cell 100 |
| 121 | Membrane that separates the region 110 for a gas sample |
| | from an electrolyte in the measuring chamber |
| 130 | Wall of the measuring chamber of the sensor cell 100 |
| 140 | Interior region of the housing 24, accommodates a power |
| | supply unit and a signal-processing sensor evaluation unit |
| 150 | Holder, holds the feed unit 10 and surrounds the wall 130 of |
| | the sensor cell 100 |
| ARA, | Exhaust gas purification system, removes ammonia from |
| ARA′ | raw gas and thereby provides clean gas, is monitored by the |
| | monitoring unit Ue.1, Ue.2, each serves as a monitored |
| | system |
| Auf | Collection container, which collects the cleaning liquid with |
| | the ammonium sulfate, belongs to the exhaust gas |
| | purification system ARA |
| F_Zr.1, | Error period in the monitoring period U_Zr |
| F_Zr.2, | |
| . . . | |
| Gb | Building which houses the stable St, the technical room Tr, |
| | and the exhaust gas purification system with the parts WW, |
| | Auf |
| LB, | Agricultural enterprise, comprises the exhaust gas |
| LB′ | purification system ARA, ARA′ and the monitoring unit Ue.1, |
| | Ue.2 |
| LW | Conductivity sensor that measures the electrical conductivity |
| | of the cleaning liquid in the collection container Auf |
| pH | pH sensor that measures the pH value of the cleaning liquid |
| | in the collection container Auf |
| Roh.1, | Region, in which raw gas can accumulate, between the |
| Roh.2 | stable St.1, St.2 and the exhaust gas purification system |
| | ARA |
| Roh′ | Region in the agricultural enterprise LB′ where raw gas can |
| | accumulate |
| St, St.1, | Stable for animal breeding |
| St.2 | |
| StE | Deaeration for the stable St, comprises the first fan Vent1 |
| StB | Aeration for the stable St |
| Tr | Technical room |
| Ue.1 | First monitoring unit, comprises the raw gas sensors 1.i1, |
| | 1.i2, the clean gas sensors 1.o1, 1.o2, the geoposition |
| | sensor 19, the volume flow sensor 12, the communication |
| | unit 2, the data memory 14 and the output unit 16, monitors |
| | the exhaust gas purification system ARA |
| Ue.2 | Second monitoring unit, comprises the raw gas sensor 1′.i1, |
| | the clean gas sensors 1′.o1, 1′.o2, the geoposition sensor |
| | 19′, the volume flow sensor 12′, the communication unit 2′ |
| | and the data memory 14′, monitors the exhaust gas |
| | purification system ARA′ |
| U Zr | Monitoring period |
| Vb | Swirl region in which the raw gas sensor 1.i1 is arranged |
| Vent1 | First fan, arranged in the deaeration region StE, extracts raw |
| | gas from the stable St |
| Vent2 | Second fan, arranged on the roof of the building Gb, |
| | extracts clean gas from the building Gb |
| WW | Scrubber wall of the chemical exhaust gas purification |
| | system ARA, adds sulfuric acid to the raw gas, belongs to |
| | the exhaust gas purification system ARA |

1-9. (canceled)

10. A monitoring assembly for monitoring a first system for ammonia emissions, wherein the monitoring assembly comprises:
a first monitoring unit; and
a central computer,
wherein the first monitoring unit comprises:
a first ammonia sensor;
a first data memory; and
a first communication unit,
wherein the first data memory stores:
a predetermined first upper limit value; and
a unique identifier of the first ammonia sensor,
wherein the first upper limit value stored in the first data memory specifies an upper limit for permissible ammonia emissions of the first system,
wherein the central computer:
is arranged spatially spaced apart from the first monitoring unit and spatially spaced apart from the first system; and
comprises a signal-processing evaluation unit,
wherein the first ammonia sensor is configured to measure the ammonia content in a gas mixture emitted by the first system,
wherein the first communication unit:
has at least temporary read access to the first data memory, and
is configured to generate a message, wherein the message is generated by the first communication unit and comprises:
the ammonia content measured by the first ammonia sensor;
the stored identifier of the first ammonia sensor; and
the first upper limit value stored in the first data memory,
wherein the monitoring assembly is configured to transmit the message generated by the first communication unit to the central computer,
wherein the evaluation unit is configured to automatically determine whether actual ammonia emissions of the first system exceed the stored first upper limit value for the first system, and
wherein the evaluation unit is further configured to determine whether the actual ammonia emissions of the first system exceed the stored first upper limit value for the first system by using the measured ammonia content and the stored first upper limit value, the stored first upper limit value and the measured ammonia content having been transmitted as part of the message from the first communication unit to the central computer.

11. The monitoring assembly of claim 10, wherein:
the monitoring assembly comprises a second monitoring unit, the second monitoring unit comprising:
a second ammonia sensor;
a second data memory; and
a second communication unit,
wherein the second ammonia sensor is configured to measure the ammonia content in a gas mixture emitted by a second system,
wherein the second data memory stores:
a predetermined second upper limit value; and
a unique identifier of the second ammonia sensor,
wherein the second upper limit value stored in the second data memory specifies an upper limit for the permissible ammonia emissions of the second system,
wherein the central computer is arranged spatially spaced apart from the second monitoring unit and spatially spaced apart from the second system,
wherein the second communication unit:
has at least temporary read access to the second data memory; and
is configured to generate a second message, wherein the second message comprises:
the ammonia content measured by the second ammonia sensor;
the stored identifier of the second ammonia sensor; and
the second upper limit value stored in the second data memory,
wherein the monitoring assembly is configured to transmit the second message to the central computer,
wherein the evaluation unit is configured to determine whether actual ammonia emissions of the second system exceed the second upper limit value, and
wherein the evaluation unit is configured to determine whether the actual ammonia emissions of the second system exceed the second upper limit value by using the ammonia content measured by the second ammonia sensor and the stored second upper limit value, the ammonia content measured by the second ammonia sensor and the stored second upper limit value having been transmitted as part of the second message.

12. The monitoring assembly of claim 10, wherein:
the first upper limit value, stored in the first data memory and transmitted as part of the message to the central computer, specifies an upper limit for the maximum amount of ammonia that the first system may emit in a predetermined reference time period,
a monitoring period is predetermined, wherein the monitoring period is at least as long as a reference time period,
the evaluation unit is configured to determine, using the message from the first communication unit, each error period in the monitoring period, wherein an error period is a period:
that is at least as long as the reference time period, and
in which the first system emits a quantity of ammonia per reference time period that exceeds the first upper limit value.

13. The monitoring assembly of claim 10, wherein:
the first monitoring unit comprises a first geoposition sensor, and
a computer-evaluable description of a first geoposition region is stored in the first data memory, wherein the stored description is such that each location of the first system is positioned in the first geoposition region,
wherein the first geoposition sensor is configured to measure a current geoposition of the first ammonia sensor,
wherein the first communication unit is configured to generate the message such that the message additionally comprises the measured current geoposition of the first ammonia sensor, and
wherein the evaluation unit is configured to determine, using the computer-evaluable description, whether the transmitted geoposition is in the first geoposition region.

14. The monitoring assembly of claim 13, wherein:
the first communication unit is configured to generate the message in such a way that the message additionally comprises the computer-evaluable description of the first geoposition region.

15. The monitoring assembly of claim 10, wherein:
the monitoring assembly comprises a first signal transmitter, wherein the first signal transmitter is configured to:
acquire a content value for an ammonia content by acquiring a given content value or by measuring, with the first signal transmitter, a content value in a gas sample; and
generate a signal comprising information of the ammonia content value acquired by the first signal transmitter,
wherein the monitoring assembly is selectively operable in:
a monitoring mode, or
a checking mode,
wherein the monitoring assembly is configured to use the first ammonia sensor when operating in the monitoring mode, and
wherein the monitoring assembly is configured to, when operating in the checking mode:
use the first signal transmitter in place of the first ammonia sensor; and
transmit the signal generated by the first signal transmitter to the central computer, and
wherein the evaluation unit is configured to, when operating in the checking mode:
determine, using the transmitted signal of the first signal transmitter, the acquired value that was acquired by the first signal transmitter.

16. A monitoring method for monitoring a first system for ammonia emissions, wherein the method is performed using a monitoring assembly:
wherein the monitoring assembly comprises:
a first monitoring unit; and
a central computer,
wherein the first monitoring unit comprises:
a first ammonia sensor;
a first data memory; and
a first communication unit,
wherein the first data memory stores:
a predetermined first upper limit value; and
a unique identifier of the first ammonia sensor,
wherein the first upper limit value stored in the first data memory specifies an upper limit for permissible ammonia emissions of the first system,
wherein the first communication unit has at least temporary read access to the first data memory,
wherein the central computer:
is arranged spatially spaced apart from the first monitoring unit and spatially spaced apart from the first system; and
comprises a signal-processing evaluation unit,
wherein the method comprises the automatically performed steps of:
with the first ammonia sensor, measuring the ammonia content in a gas mixture emitted by the first system;
with the first communication unit, generating a message comprising:
an ammonia content measured by the first ammonia sensor;
the stored identifier of the first ammonia sensor;
the first upper limit value stored in the first data memory;
transmitting, to the central computer, the message generated by the first communication unit; and
with the evaluation unit, automatically determining whether actual ammonia emissions of the first system exceed the stored first upper limit value for the first system,
wherein the evaluation unit is configured to determine whether the actual ammonia emissions of the first system exceed the stored first upper limit value for the first system by using the measured ammonia content and the stored first upper limit value, the stored first upper limit value and the measured ammonia content having been transmitted as part of the message from the first communication unit to the central computer.

17. The monitoring method of claim 16, wherein:
the monitoring assembly comprises a second monitoring unit, the second monitoring unit comprising:
a second ammonia sensor;
a second data memory; and
a second communication unit,
wherein the second ammonia sensor is configured to measure the ammonia content in a gas mixture emitted by a second system,
wherein the method further comprises:
storing, in the second data memory:
a predetermined second upper limit value; and
a unique identifier of the second ammonia sensor,
wherein the second upper limit value stored in the second data memory specifies an upper limit for the permissible ammonia emissions of the second system,
wherein the central computer is arranged spatially spaced apart from the second monitoring unit and spatially spaced apart from the second system,
wherein the second communication unit has at least temporary read access to the second data memory,
wherein the method further comprises:
generating, with the second communication unit, a second message, wherein the second message comprises:
the ammonia content measured by the second ammonia sensor;
the stored identifier of the second ammonia sensor; and
the second upper limit value stored in the second data memory;
transmitting, to the central computer, the second message; and
determining, with the evaluation unit, whether actual ammonia emissions of the second system exceed the second upper limit value,
wherein the determining whether the actual ammonia emissions of the second system exceed the second upper limit value comprises using the ammonia content measured by the second ammonia sensor and the stored second upper limit value, the ammonia content measured by the second ammonia sensor and the stored second upper limit value having been transmitted as part of the second message.

18. The monitoring method of claim 16, wherein:
the first upper limit value, stored in the first data memory and transmitted as part of the message to the central computer, specifies an upper limit for the maximum amount of ammonia that the first system may emit in a predetermined reference time period,
the method further comprising:
predetermining a monitoring period, wherein the monitoring period is at least as long as a reference time period,
determining, by the evaluation unit and using the message transmitted from the first communication unit, each error period in the monitoring period, wherein an error period is a period:
that is at least as long as the reference time period, and
in which the first system emits a quantity of ammonia per reference time period that exceeds the first upper limit value.

19. The monitoring method of claim 16, wherein
the first monitoring unit comprises a first geoposition sensor,
wherein the method further comprises:
storing, in the first data memory, a computer-evaluable description of a first geoposition region, wherein the stored description is such that each location of the first system is positioned in the first geoposition region;
with the first geoposition sensor, measuring a current geoposition of the first ammonia sensor;
with the first communication unit, generating the message such that the message additionally comprises the measured geoposition of the first ammonia sensor; and
with the evaluation unit and using the computer-evaluable description, determining whether the transmitted geoposition is in the first geoposition region.

20. The monitoring method of claim 19, wherein the first communication unit is configured to generate the message in such a way that the message additionally comprises the computer-evaluable description of the first geoposition region.

21. The monitoring method of claim 16, wherein:
the monitoring assembly comprises a first signal transmitter,
the monitoring assembly is checked by means of a checking method, wherein the checking method comprises:
using the first signal transmitter in place of the first ammonia sensor;
with the first signal transmitter, acquiring a content value for an ammonia content by acquiring a given content value or by measuring, with the first signal transmitter, a content value in a gas sample,
with the first signal transmitter, generating a signal such that the generated signal comprises information about the ammonia content;
transmitting the signal generated by the first signal transmitter to the central computer; and
with the evaluation unit and based on the received signal, determining the content value for the ammonia content that was acquired by the first signal transmitter.
